(19) United States
(12) Patent Application Publication
(10) Pub. No.: US 2026/0151754 A1
(43) Pub. Date: Jun. 4, 2026

(54) A CATALYST FOR CO2 HYDROGENATION TO C2+ ALCOHOLS, PREPARATION METHOD AND APPLICATION THEREOF

(71) Applicant: Yankuang Energy R&D Co., LTD, Shanghai (CN)

(72) Inventors: Qiwen Sun, Shanghai (CN); Haibing Sheng, Shanghai (CN); Yan Sun, Shanghai (CN); Zongsen Zhang, Shanghai (CN); Chuansong Li, Shanghai (CN); Junyang Ma, Shanghai (CN)

(73) Assignee: Yankuang Energy R&D Co., LTD, Shanghai (CN)

(21) Appl. No.: 18/877,353

(22) PCT Filed: Oct. 30, 2023
(86) PCT No.: PCT/CN2023/127459

§ 371 Date: Dec. 20, 2024

(30) Foreign Application Priority Data
May 22, 2023 (CN) 202310575287.X

(51) Int. Cl. B01J 23/889; B01J 35/33; B01J 37/02; B01J 37/03; B01J 37/04; B01J 37/06; B01J 37/08; B01J 37/18; C07C 1/12

(52) U.S. Cl. CPC B01J 23/8892; B01J 35/33; B01J 37/0236; B01J 37/031; B01J 37/04; B01J 37/06; B01J 37/088; B01J 37/18; C07C 1/12

(57) ABSTRACT

A catalyst for CO₂ hydrogenation to C₂₊ alcohols is disclosed. Active components of the catalyst comprise Fe, Cu and M, wherein M comprises one or more of Zn, Mn, Co, Cs, Ce, Ga, Al and Zr. The molar ratio of Fe, Cu and M is Fe:Cu:M=1:(1˜4):(0.5˜4). The application also provides a preparation method for the catalyst. The preparation is simple, and the raw materials are cheap and easily available. By adjusting the proportions of different active components, efficient synergy between the components is achieved, and CO₂ conversion rate and C₂₊ alcohols selectivity are improved. The application also provides the application of the catalyst, which achieves CO₂ conversion rate ≥35%, C₂₊ alcohols selectivity ≥15% and CH₄ selectivity ≤15% in catalyzing the CO₂ hydrogenation to C₂₊ alcohols. The catalyst increases the content of high value-added C₂₊ alcohols in the products of CO₂ hydrogenation to alcohols.

### FIELD OF THE DISCLOSURE

The application relates to the technical field of carbon dioxide conversion, and further to a catalyst for CO₂ hydrogenation to C₂₊ alcohols, preparation method and application thereof.

### BACKGROUND

The utilization of carbon dioxide has an important impact on the development of carbon cycle and circular economy, and its catalytic conversion has attracted worldwide attention. Among the products of many carbon dioxide conversions, methanol is the main product, while there are fewer studies on carbon dioxide hydrogenation to C₂₊ alcohols. C₂₊ alcohols are not only non-toxic but also a more valuable product, which can be easily converted into high value-added chemicals. For the CO₂ hydrogenation to C₂₊ alcohols, it is necessary to develop catalysts with high activity and high selectivity for C₂₊ alcohols. At present, it is still very challenging to obtain catalysts with relatively high or absolute selectivity for C₂₊ alcohols at relatively high carbon dioxide conversion levels.

Therefore, finding a catalyst with high stability and high efficiency to catalyze the CO₂ hydrogenation to C₂₊ alcohols, is very important for the application and conversion of carbon dioxide. At present, the relatively mature catalysts in the technology of CO₂ hydrogenation to alcohols are usually derived from the catalyst of CO hydrogenation to methanol. From the distribution of the catalytic reaction products, CO₂ is mainly converted into methanol after hydrogenation, and there is also a small amount of organic product methane. In addition, since the catalyst can simultaneously promote the reverse water gas shift reaction, a part of CO₂ will also be converted into CO.

Patent CN111659432A invented an iron-based catalyst, preparation method and application for CO₂ hydrogenation to ethanol. The active sites of the iron-based catalyst for CO₂ hydrogenation to ethanol comprise Fe₅C₂, Fe₂C and Fe₃C, and the catalyst has an ethanol selectivity ≥20% and a CO selectivity ≤10% during the CO₂ hydrogenation to ethanol. Thongthai Witoon et al. reported a new K—Co/In₂O₃ catalyst for CO₂ hydrogenation to C₂₊ alcohols. When the proportions of K and Co are 2.5 wt % and 5.0 wt % respectively, the optimal space-time yield of C₂₊ alcohols is 169.6 gkgcat⁻¹ h⁻¹. Characterization results show that the K—O—Co species formed in the catalyst significantly reduce the number of weak H₂ adsorption sites and enhance the interactions of the adsorbed H. The weak H₂ reductive adsorption and the improved interactions between the adsorbed H and the catalyst surface slow down the hydrogenation ability, promoting the insertion of CO into the adsorbed CxHy species before its hydrogenation to hydrocarbons. This leads to a significant reduction in CH₄ and higher hydrocarbons, while significantly increasing the selectivity for higher alcohols.

It is well known that compared to methanol, C₂₊ alcohols such as ethanol and propanol are more valuable chemicals. Moreover, high-carbon alcohols containing C₂₊ species are also superior gasoline additives compared to methanol. Therefore, it is necessary to develop new catalysts for CO₂ hydrogenation to C₂₊ alcohols with high selectivity and high CO₂ conversion efficiency.

### SUMMARY OF THE INVENTION

The present application aims to address the issues of low CO₂ conversion rate, low C₂₊ selectivity, and high CH₄ selectivity in the existing catalysts for CO₂ hydrogenation to C₂₊ alcohols. The objective is to provide a catalyst for CO₂ hydrogenation to C₂₊ alcohols, preparation method and application thereof. The catalyst provided by the present application achieves high-efficiency synergistic catalytic effects between the active components by adjusting the proportions of different metal active components. The catalyst prepared by the application achieves a CO₂ conversion rate of ≥40%, a C₂₊ alcohols selectivity of ≥20%, and a CH₄ selectivity of ≤10% in the CO₂ hydrogenation to C₂₊ alcohols process.

In order to achieve the above-mentioned object, the present application provides a catalyst for CO₂ hydrogenation to C₂₊ alcohols, active components of the catalyst comprise Fe, Cu and M, wherein M comprises one or more of Zn, Mn, Co, Cs, Ce, Ga, Al and Zr; and a molar ratio of Fe, Cu and M is Fe:Cu:M=1:(1˜4):(0.5˜4).

The present application further provides a preparation method for the catalyst for CO₂ hydrogenation to C₂₊ alcohols, which comprises following steps:
S1: measuring metal salt solutions containing Fe, Cu and M to prepare a mixed salt solution according to a composition ratio of the catalyst, and weighting a precipitant to prepare a precipitant solution;
S2: adding the mixed salt solution and the precipitant solution into a container in parallel to perform a coprecipitation reaction to obtain a reaction mixture, and aging the reaction mixture;
S3: washing, drying and calcining the reaction mixture after aging to obtain the catalyst.

Preferably, the step S3 further comprises conducting a conductivity test on a filtrate after washing, washing the reaction mixture until a conductivity of the filtrate is 200˜3000 μS/cm, and then performing the drying and calcining.

In some embodiments, the metal salt solution containing Fe is ferric nitrate solution; the metal salt solution containing Cu is copper nitrate solution; the metal salt containing M is a solution which is made from one or a mixture of zinc nitrate, manganese nitrate, cobalt nitrate, cesium nitrate, cerium nitrate, aluminum nitrate, and zirconium nitrate.

In some embodiments, the precipitant is one or a mixture of potassium carbonate, sodium carbonate and ammonium carbonate.

In some embodiments, a reaction temperature of the coprecipitation reaction is 60˜80° C.; a pH value of the reaction mixture is 6.0˜8.0; an aging temperature is 60˜80° C. and an aging duration is 0.5˜4 h; a drying temperature is 110° C. and a drying duration is 12 h; a calcining temperature is 450° C. and a calcining duration is 4 h.

The present application further provides an application of a catalyst for CO₂ hydrogenation to C₂₊ alcohols, which comprises pre-reducing the catalyst according to claim 1, and using the catalyst after pre-reducing to catalyze the CO₂ hydrogenation to C₂₊ alcohols.

In some embodiments, conditions of the pre-reducing are: a reducing gas is H₂, a space velocity of the reducing gas is 2000 mL/g/h, a reducing pressure is 0.5 MPa, a reducing temperature is 400° C., a reducing duration is 2 h, and a heating rate is 2° C./min.

In some embodiments, conditions for using the catalyst in the CO₂ hydrogenation to C₂₊ alcohols are: reaction atmospheres are H₂ and CO₂, a reaction pressure is 2 MPa˜6 MPa, and a reaction temperature is 280° C.˜400° C.

In some embodiments, the ratio of H₂ to CO₂ in the reaction atmospheres is H₂/CO₂=2, H₂/CO₂=3 or H₂/CO₂=4.

Compared with the prior art, the present application can bring the following beneficial effects:
1. The active components of the catalyst provided by the present application comprise Fe and Cu. Fe exhibits strong Fischer-Tropsch reaction activity, while Cu has excellent CO adsorption and insertion capability. The combination of these two metals enables the catalyst to achieve high activity in CO₂ hydrogenation to C₂₊ alcohols. The active components of the catalyst further comprise active metals such as Zn and Mn, which can further enhance CO adsorption, insertion ability, and hydrogenation performance. Taking Zn and Mn as examples, Mn combines with Fe to form the Fe-Mn-O phase, which improves CO adsorption capacity and promotes the dispersion of Fe and Cu active metal particles. Zn enhances hydrogenation performance and increases active intermediates such as CH*, CH₂*, and CH₃*, thereby improving alcohols selectivity. By adjusting the proportions of the active components, the catalyst achieves efficient synergistic effects, significantly improving CO₂ conversion and C₂₊ alcohols selectivity.
2. The catalyst provided by the present application has high mechanical strength and better dispersion of active components, enabling long-term stable operation in reactors.
3. Additionally, the catalyst provided by the present application features uniform composition, low-cost and readily available raw materials, simple preparation methods, and short preparation cycles, making it suitable for large-scale industrial production.

### BRIEF DESCRIPTION OF THE DRAWINGS

FIG. 1 is a scanning electron microscope (SEM) image of the catalyst for CO₂ hydrogenation to C₂₊ alcohols provided by the present application.

### DESCRIPTION OF THE ILLUSTRATED EMBODIMENTS

The following will be combined with specific embodiments to clearly and completely describe the technical solutions in the present application. The following embodiments are only used to more clearly illustrate the technical solutions of the present application, and cannot be used to limit the protection scope of the present application.

### Embodiment 1

The present application provides a catalyst for CO₂ hydrogenation to C₂₊ alcohols. The active components of the catalyst comprise Fe, Cu and M, wherein M comprises one or more of Zn, Mn, Co, Cs, Ce, Ga, Al and Zr.

Among them, the molar ratio of Fe, Cu and M is Fe:Cu:M=1:1˜4:0.5˜4.

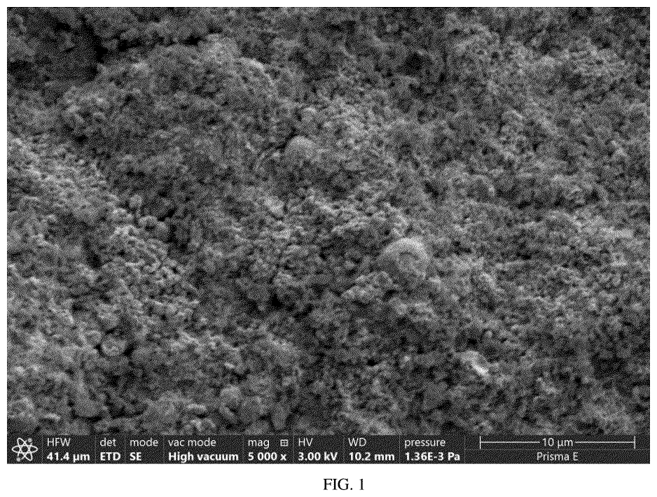

FIG. 1 shows the scanning electron microscope image of the catalyst.

### Embodiment 2

Based on Embodiment 1, the present application provides a preparation method for the catalyst, and the specific steps are as follows:
S1: measuring metal salt solutions containing Fe, Cu and M to prepare a mixed salt solution according to a composition ratio of the catalyst, and weighting a precipitant to prepare a precipitant solution;
S2: adding the mixed salt solution and the precipitant solution into a container in parallel to perform a coprecipitation reaction to obtain a reaction mixture, and aging the reaction mixture;
S3: washing, drying and calcining the reaction mixture after aging to obtain the catalyst.

In step S1, the metal salt solution containing Fe is ferric nitrate solution; the metal salt solution containing Cu is copper nitrate solution; the metal salt containing M is a solution which is made from one or a mixture of zinc nitrate, manganese nitrate, cobalt nitrate, cesium nitrate, lanthanum nitrate, cerium nitrate, aluminum nitrate, and zirconium nitrate.

The above metal salt solutions can be first prepared into 1M metal salt solutions respectively, and the amount of the metal salt solutions containing Fe, Cu and M is calculated according to the molar ratio of the active components of the catalyst, and then the above metal salt solutions are measured and evenly mixed to prepare a mixed salt solution.

The above-mentioned precipitant can be selected from one or a mixture of potassium carbonate, sodium carbonate and ammonium carbonate.

In step S2, the flow rates of the mixed salt solution and the precipitant solution are controlled by a peristaltic pump and added into a three-neck flask equipped with a stirrer to undergo a co-precipitation reaction in parallel, resulting in a reaction mixture. The reaction temperature is maintained at 60˜80° C., and the pH of the reaction mixture is maintained at 6.0˜8.0. After the dropwise addition of the mixed salt solution and precipitant solution is completed, stirring continues at 60˜80° C. for an aging process, with an aging duration of 0.5˜4 hours.

In step S3, after the aging process, the reaction mixture is naturally cooled to 20° C. and then washed by suction filtration with deionized water. The conductivity of the filtrate is monitored using a conductivity meter. When the conductivity of the filtrate reaches 200˜3000 μS/cm, the reaction mixture is placed in an oven at 110° C. for 12 hours of drying. Subsequently, it is calcined in a muffle furnace at 450° C. for 4 hours to obtain the catalyst.

### Embodiment 3

In this embodiment, the application of the catalyst provided by the present application in the reaction of CO₂ hydrogenation to C₂₊ alcohols is described. First, the catalyst is pre-reduced, and the conditions of the pre-reducing are: a reducing gas is H₂, a space velocity of the reducing gas is 2000 mL/g/h, a reducing pressure is 0.5 MPa, a reducing temperature is 400° C., a reducing duration is 2 h, and a heating rate is 2° C./min.

The pre-reduced catalyst is then used in the CO₂ hydrogenation to C₂₊ alcohols reaction process. The catalytic reaction conditions are: reaction atmospheres are H₂ and CO₂, the ratio of H₂ to CO₂ in the reaction atmospheres is H₂/CO₂=2, H₂/CO₂=3 or H₂/CO₂=4, a reaction pressure is 2 MPa˜6 MPa, and a reaction temperature is 280° C.˜400° C.

The reactor for the CO₂ hydrogenation to C₂₊ alcohols process catalyzed by the catalyst can be one of a fixed-bed reactor, fluidized-bed reactor, or slurry-bed reactor, and its specific structure and form are not specifically limited in this embodiment.

To better understand and apply the above solution, and to effectively demonstrate its corresponding benefits, the following further elaborates on the catalyst for CO₂ hydrogenation to C₂₊ alcohols, along with its preparation method and application, in conjunction with specific embodiments.

### Embodiment 4

S1: First, dissolve copper nitrate, ferric nitrate, zinc nitrate, manganese nitrate, and potassium carbonate (precipitant) in deionized water to prepare 1M solutions. Then, take 150 mL of 1M copper nitrate solution, 75 mL of 1M ferric nitrate solution, 37.5 mL of 1M zinc nitrate solution, and 135 mL of 1M manganese nitrate solution, and mix them uniformly in a 500 mL beaker to form a mixed salt solution. In another 500 mL beaker, measure 600 mL of 1M potassium carbonate solution.
S2: adding the above mixed salt solution and potassium carbonate solution into a three-neck flask equipped with a stirrer in parallel to carry out a coprecipitation reaction, yielding a reaction mixture. Maintain the reaction temperature at 80° C., adjust the addition rate of the potassium carbonate solution using a peristaltic pump, and control the pH to 8.0. After the addition is complete, continue stirring at 80° C. for 2 hours for aging.
S3: After the aging reaction, naturally cool the reaction mixture to 20° C., then wash by suction filtration using deionized water. Measure the conductivity of the filtrate with a conductivity meter, and when it reaches 240 μS/cm, place the reaction mixture in an oven at 110° C. to dry for 12 hours. Then, calcine the mixture in a muffle furnace at 450° C. for 4 hours to obtain the KFe₁Cu₂Zn_{0.5}Mn_{1.8} catalyst.

Activity test is performed to the KFe₁Cu₂Zn_{0.5}Mn_{1.8} catalyst.

The catalyst is first pre-reduced under the following conditions: reducing gas H₂, reduction pressure of 0.5 MPa, space velocity of 2000 mL/g/h, and reduction temperature of 400° C. for 2 hours with a heating rate of 2° C./min. After reduction, the catalyst is subjected to catalytic performance evaluation under the following conditions: the H₂/CO₂ ratio in the reaction atmosphere is 3.0, space velocity is 3000 mL/g/h, reaction temperature is 300° C., and reaction pressure is 3.0 MPa. The catalyst undergoes a 50-hour activity test, and the catalytic performance results are shown in Table 1.

### Embodiment 5

Referring to Embodiment 4, the difference is that the amount of 1M manganese nitrate solution in Embodiment 4 is changed to 45 ml, and other preparation conditions are the same as Embodiment 4 to obtain KFe₁Cu₂Zn_{0.5}Mn_{0.6} catalyst.

Conditions of the activity test are the same as those in Embodiment 4. The catalyst is tested for activity for 50 hours. The catalytic performance results are shown in Table 1.

### Embodiment 6

Referring to Embodiment 4, the difference is that 37.5 ml of 1 M cobalt nitrate solution is added when preparing the mixed salt solution, and other preparation conditions are the same as those of Embodiment 4 to obtain a KCo_{0.5}Fe₁Cu₂Zn_{0.5}Mn_{1.8} catalyst.

Conditions of the activity test are the same as those in Embodiment 4. The catalyst is tested for activity for 50 hours. The catalytic performance results are shown in Table 1.

### Embodiment 7

Referring to Embodiment 4, the difference is that 37.5 ml of 1M zirconium nitrate solution is added when preparing the mixed salt solution, and other preparation conditions are the same as those of Embodiment 4 to obtain a KZr_{0.5}Fe₁Cu₂Zn_{0.5}Mn_{1.8} catalyst.

Conditions of the activity test are the same as those in Embodiment 4. The catalyst is tested for activity for 50 hours. The catalytic performance results are shown in Table 1.

### Embodiment 8

Referring to Embodiment 4, the difference is that the pH of the reaction mixture is adjusted to 7.0, and other preparation conditions are the same as those in Embodiment 4 to obtain a KFe₁Cu₂Zn_{0.5}Mn_{1.8} catalyst.

Conditions of the activity test are the same as those in Embodiment 4. The catalyst is tested for activity for 50 hours. The catalytic performance results are shown in Table 1.

### Embodiment 9

Referring to Embodiment 4, the difference is that the conductivity of the water-washed filtrate of the reaction mixture is controlled at 1300 μS/cm, and the other preparation conditions are the same as those of Embodiment 4, to obtain a KFe₁Cu₂Zn_{0.5}Mn_{1.8} catalyst.

Conditions of the activity test are the same as those in Embodiment 4. The catalyst is tested for activity for 50 hours. The catalytic performance results are shown in Table 1.

### Comparative Embodiment 1

Referring to Embodiment 4, the difference is that 1M manganese nitrate solution is not added when preparing the mixed salt solution, and other preparation conditions are the same as those of Embodiment 4 to obtain a KFe₁Cu₂Zn_{0.5} catalyst.

Conditions of the activity test are the same as those in Embodiment 4. The catalyst is tested for activity for 50 hours. The catalytic performance results are shown in Table 1.

### Comparative Embodiment 2

Referring to Embodiment 4, the difference is that 1M zinc nitrate solution is not added when preparing the mixed salt solution, and other preparation conditions are the same as those of Embodiment 4 to obtain a KFe₁Cu₂Mn_{1.8} catalyst.

Conditions of the activity test are the same as those in Embodiment 4. The catalyst is tested for activity for 50 hours. The catalytic performance results are shown in Table 1.

TABLE 1
Catalytic performance results of different catalysts in
Embodiments 4~9 and Comparative Embodiments 1~2

| | CO₂ | | Alcohol/Hydrocarbon Selectivity(%) | | | | | |
|---|---|---|---|---|---|---|---|---|
| | conversion | CO | | C₂₋₄ | C₂₋₄ | C₅₊ | | C₂₊ |
| catalyst | rate(%) | selectivity(%) | CH₄ | alkanes | olefins | hydrocarbons | methanol | alcohols |
| Embodiment 4 | 40.15 | 9.37 | 8.56 | 6.65 | 15.89 | 47.41 | 0.72 | 20.77 |
| Embodiment 5 | 35.35 | 11.72 | 14.58 | 8.58 | 18.89 | 40.57 | 1.01 | 16.37 |
| Embodiment 6 | 47.49 | 5.93 | 14.93 | 6.32 | 16.8 | 44.77 | 2.13 | 15.05 |
| Embodiment 7 | 38.70 | 9.99 | 14.93 | 12.17 | 13.94 | 41.96 | 1.96 | 15.04 |
| Embodiment 8 | 35.42 | 10.25 | 13.21 | 10.21 | 13.42 | 44.21 | 2.01 | 16.94 |
| Embodiment 9 | 36.21 | 11.48 | 14.86 | 9.74 | 14.58 | 43.56 | 1.87 | 15.39 |
| Comparative | 36.37 | 11.63 | 13.69 | 31.98 | 2.94 | 36.86 | 1.99 | 12.54 |
| Embodiment 1 | | | | | | | | |
| Comparative | 28.96 | 20.56 | 9.57 | 6.22 | 10.95 | 55.47 | 1.15 | 16.64 |
| Embodiment 2 | | | | | | | | |

As shown in Table 1, the catalyst obtained in Embodiment 4 is different from that in Comparative Embodiment 1 in that the active component Mn is added during the catalyst preparation process. From the catalytic performance results of the two, it can be seen that the CO₂ conversion rate and C₂₊ alcohols selectivity of Embodiment 4 are higher than those of Comparative Embodiment 1, and the selectivity of alcohols and hydrocarbons such as CO, CH₄, C₂₋₄ alkanes and methanol are lower than those of Comparative Embodiment 1.

The catalyst obtained in Embodiment 4 is different from that in Comparative Embodiment 2 in that the active component Zn is added during the catalyst preparation process. From the catalytic performance results of the two, it can be seen that the CO₂ conversion rate and C₂₊ alcohols selectivity of Embodiment 4 are higher than those of Comparative Embodiment 2, and the selectivity of alcohols and hydrocarbons such as CO, CH₄, C₅₊ hydrocarbons and methanol are lower than those of Comparative Embodiment 1.

Combined with Embodiments 4-9, the catalysts provided in the above embodiments all achieved catalytic effects of CO₂ conversion rate ≥35%, C₂₊ alcohols selectivity ≥15%, and CH₄ selectivity ≤15%, showing good application potential for CO₂ hydrogenation to C₂₊ alcohols.

Specific embodiments are used herein to illustrate the principles and implementation methods of the present application. The description of the above embodiments is only used to help understand the method of the present application and its core idea, comprising the best mode, and also enables any technician in the field to practice the present application, including the manufacture and use of any device or system, and the implementation of any combined method.

It should be pointed out that for ordinary technicians in this technical field, several improvements and modifications can be made without departing from the principles of the present application, and these improvements and modifications should also be regarded as within the scope of protection of the present application.

1. A catalyst for CO₂ hydrogenation to C₂₊ alcohols, characterized in that:
active components of the catalyst comprise Fe, Cu and M, wherein M comprises one or more of Zn, Mn, Co, Cs, Ce, Ga, Al and Zr;
and a molar ratio of Fe, Cu and M is Fe:Cu:M=1:(1˜4):(0.5˜4).

2. A preparation method for the catalyst for CO₂ hydrogenation to C₂₊ alcohols according to claim 1, characterized by comprising following steps:
S1: measuring metal salt solutions containing Fe, Cu and M to prepare a mixed salt solution according to a composition ratio of the catalyst, and weighting a precipitant to prepare a precipitant solution;
S2: adding the mixed salt solution and the precipitant solution into a container in parallel to perform a coprecipitation reaction to obtain a reaction mixture, and aging the reaction mixture;
S3: washing, drying and calcining the reaction mixture after aging to obtain the catalyst.

3. The preparation method according to claim 2, characterized in that:
the step S3 further comprises conducting a conductivity test on a filtrate after washing, washing the reaction mixture until a conductivity of the filtrate is 200˜3000 μS/cm, and then performing the drying and calcining.

4. The preparation method according to claim 2, characterized in that:
the metal salt solution containing Fe is ferric nitrate solution;
the metal salt solution containing Cu is copper nitrate solution;
the metal salt solution containing M is a solution which is made from one or a mixture of zinc nitrate, manganese nitrate, cobalt nitrate, cesium nitrate, cerium nitrate, aluminum nitrate, and zirconium nitrate.

5. The preparation method according to claim 2, characterized in that:
the precipitant is one or a mixture of potassium carbonate, sodium carbonate and ammonium carbonate.

6. The preparation method according to claim 2, characterized in that:
a reaction temperature of the coprecipitation reaction is 60˜80° C.;
a pH value of the reaction mixture is 6.0˜8.0;
an aging temperature is 60˜80° C. and an aging duration is 0.5˜4 h;
a drying temperature is 110° C. and a drying duration is 12 h;
a calcining temperature is 450° C. and a calcining duration is 4 h.

7. An application of a catalyst for CO₂ hydrogenation to C₂₊ alcohols, characterized in that:
pre-reducing the catalyst according to claim 1, and using the catalyst after pre-reducing to catalyze the CO₂ hydrogenation to C₂₊ alcohols.

8. The application according to claim 7, characterized in that:
conditions of the pre-reducing are: a reducing gas is H₂, a space velocity of the reducing gas is 2000 mL/g/h, a reducing pressure is 0.5 MPa, a reducing temperature is 400° C., a reducing duration is 2 h, and a heating rate is 2° C./min.

9. The application according to claim 7, characterized in that:
conditions for using the catalyst in the CO₂ hydrogenation to C₂₊ alcohols are: reaction atmospheres are H₂ and CO₂, a reaction pressure is 2 MPa˜6 MPa, and a reaction temperature is 280° C.˜400° C.

10. The application according to claim 9, characterized in that:
the ratio of H₂ to CO₂ in the reaction atmospheres is H₂/CO₂=2, H₂/CO₂=3 or H₂/CO₂=4.
